(19) United States
(12) Patent Application Publication
(10) Pub. No.: US 2021/0125709 A1
(43) Pub. Date: Apr. 29, 2021

(54) SYSTEM AND METHOD TO VISUALIZE AND COORDINATE IMAGE ACQUISITION WORKFLOWS

(71) Applicant: KONINKLIJKE PHILIPS N.V., EINDHOVEN (NL)

(72) Inventors: Yuechen QIAN, LEXINGTON, MA (US); Ranjith Naveen TELLIS, TEWKSBURY, MA (US); Sandeep Madhukar DALAL, WINCHESTER, MA (US); Olga STAROBINETS, NEWTON, MA (US); Hareesh CHAMARTHI, CAMBRIDGE, MA (US); Vjay PARTHASARATHY, LEXINGTON, MA (US)

(21) Appl. No.: 17/069,895

(22) Filed: Oct. 14, 2020

Related U.S. Application Data
(60) Provisional application No. 62/924,722, filed on Oct. 23, 2019.

(51) Int. Cl. G16H 40/20; G16H 30/40

(52) U.S. Cl. CPC G16H 40/20; G16H 30/40

(57) ABSTRACT

An apparatus (10) includes at least one electronic processor (20) programmed to: display, on a display device (24), at least one workflow (27) corresponding to an imaging examination order (11) for a patient, the workflow including a set of phases (31) over predicted time windows for the workflow, each phase in the set of phases including one or more steps (33) to be performed in the workflow; and dynamically update the at least one workflow on the display device with additional data related to one or more steps of the phases of the at least one workflow.

### CROSS REFERENCE TO RELATED APPLICATIONS

This application claims the benefit of U.S. Provisional Patent Application No. 62/924,722 filed Oct. 23, 2019. This application is hereby incorporated by reference herein.

### FIELD

The following relates generally to the imaging arts, imaging workflow arts, imaging workflow generation and visualization arts, and related arts.

### BACKGROUND

Radiological imaging sessions require the coordination of radiologists, technologists, other clinical medical staff (e.g., nurses, anesthesiologists, and so forth), non-clinical staff (e.g., schedulers, transport team, and so forth) and the patient, in order to acquire high-quality medical images for diagnosis, optimize utilization of the available medical imaging devices, reduce unnecessary interruptions, avoid unnecessary no-shows or repeated exams, and shorten the imaging examination turn-around time.

Imaging facilities have protocols and standard operating procedures that staff follow in order to perform imaging exams. Performing an imaging exam entails multiple steps, typically including: patient registration, patient preparation (administering anesthesia and/or an exogenous contrast agent and/or a radiopharmaceutical), patient positioning on the imaging examination table and loading into the imaging scanner, acquiring a scout imaging sequence, acquiring diagnostic imaging sequences, review and confirmation of image quality, post-processing, patient exit, and room preparation.

These steps may have various time constraints. For example, there may be a required time interval between administration of a radiopharmaceutical and commencement of imaging, in order to allow for the radiopharmaceutical to concentrate in the organ or tissue of interest. In the case of an imaging sequence designed to observe contrast agent inflow and washout, the administration of the contrast agent is timed with the imaging sequence to ensure that the imaging captures these dynamics. Furthermore, steps such as patient preparation may be dependent upon the physical health, size, or other characteristics of the patient. For example, patient preparation for an obese patient may be different from patient preparation for an underweight patient; likewise, preparation for a wheelchair-bound patient may entail additional sub-steps such as the need for one or more transport team members to assist in moving the patient from the wheelchair to the table.

Technologists, who operate the medical imaging device to acquire the medical images, are key to the whole image acquisition workflow, while other staff need to be available when needed in each step. Late arrivals by patients can lead to cancelling of the appointment, which results in the under-utilization of clinical resources (e.g., image scanners and staff). If the patient is not ready for image acquisition (e.g. due to lack of prior patient information, patient education, etc.), the exam can take longer time than planned and might even be aborted. Moreover, if a radiologist needs to check the view/quality of image, the technologist preferably timely informs the radiologist to reduce interruptions and unnecessary waiting for radiologists.

Leveraging real-time information about the status of imaging exams by medical personnel who are orchestrating the workflows on the floor or remotely in a command center, is challenging. Existing workflow management technologies typically provide information on the scheduled start and end times of scheduled medical imaging examinations, along with some additional information such as patient name, imaging modality, or so forth.

The following discloses certain improvements to overcome these problems and others.

### SUMMARY

In one aspect, an apparatus includes at least one electronic processor programmed to: display, on a display device, at least one workflow corresponding to an imaging examination order for a patient, the workflow including a set of phases over predicted time windows for the workflow, each phase in the set of phases including one or more steps to be performed in the workflow; and dynamically update the at least one workflow on the display device with additional data related to one or more steps of the phases of the at least one workflow.

In another aspect, an apparatus includes at least one electronic processor programmed to: display, on a display device, at least one workflow corresponding to an imaging examination order for a patient, the workflow including a set of phases over predicted time windows for the workflow, each phase in the set of phases including one or more steps to be performed in the workflow; and retrieve a template for a type of imaging examination to which the imaging examination order corresponds, the at least one workflow comprising or being based on the template.

In another aspect, a method includes: displaying, on a display device, at least one workflow corresponding to an imaging examination order for a patient, the workflow including a set of phases over predicted time windows for the workflow, each phase in the set of phases including one or more steps to be performed in the workflow; retrieving a template for a type of imaging examination to which the imaging examination order corresponds, the at least one workflow comprising or being based on the template; dynamically updating the at least one workflow on the display device with additional data from the retrieved template; and outputting one or more alerts or reminders when one or more of the steps of phases of the at least one workflow is completed or will soon be completed based on the dynamically updated at least one workflow.

One advantage resides in providing an imaging examination workflow visualization displaying phases or steps in the examination, along with information on the current phase or step.

Another advantage resides in providing an imaging examination workflow visualization displaying how much time until a next phase or step in the examination.

Another advantage resides in providing an imaging examination workflow visualization that is dynamically adjusted to reflect, in real-time, changes to the scheduled imaging examination workflow.

Another advantage resides in providing an imaging examination workflow visualization that proactively issues alerts to relevant medical personnel in advance of an upcoming phase or step of the imaging examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for generating an imaging examination workflow visualization in accordance with the present disclosure.

FIG. 2 shows an example of a template for use by the apparatus of FIG. 1.

FIGS. 3A-3D show examples of workflows displayed on the apparatus of FIG. 1.

### DETAILED DESCRIPTION

Existing imaging workflow visualization systems operate at a high level, usually providing a graph as a function of time blocking off imaging procedures with each block labeled by basic information such as patient name, imaging modality, and anatomical part. This provides little guidance for assisting in scheduling assistive staff, radiologist availability, use of shared ancillary equipment, use of shared patient preparation spaces, or so forth.

The following discloses an improved patient imaging workflow visualization, which represents the phases of each imaging examination workflow with additional information for each phase. To do so, a database of imaging templates is maintained for different types of imaging procedures. Each template stores information at a high degree of granularity, including defining a set of phases of the procedure (e.g., patient registration, patient waiting, intravenous (IV) therapy placement, entry into scanning room, start of scanning, end of scanning, patient discharge waiting, room cleanup) and the personnel and ancillary equipment needed (or possibly needed) for each phase. The templates may be in business process model and notation (BPMN) format, an extensible markup language (XML) format, or some other format that enables storing this information in a time flow format.

For each patient, the appropriate template is retrieved based on information provided in the examination order. The template is instantiated for the patient, for example by filling in actual (scheduled) times for the various phases, adding or removing phases as appropriate for the specific patient (which may entail retrieving/initiating additional “sub”-templates, for example a sub-template for transferring a patient from a wheelchair to the imaging transport, or for switching a patient from a general infusion pump to an MR-compatible infusion pump), adding patient-specific information such as head coil size (based on patient gender information or physical measurements if available), and/or so forth. The patient-specific instantiation may also utilize historical data, present workload and/or staff levels to adjust the predicted time intervals for various phases.

In some embodiments, the patient-specific instantiation may also include identifying and associating a specific imaging scanner to the patient-specific imaging workflow. For example, the instantiation may reference an imaging scanners list that is dynamically updated to indicate, for each schedule time slot, whether a given imaging scanner is available, not-in-use (for example, because it is undergoing maintenance), or is assigned (and in the latter case to which imaging examination it is assigned).

In some embodiments, the patient-specific instantiation may also include identifying and associating specific ancillary equipment to the patient-specific imaging workflow. For example, the template for a brain MRI scan may specify a head coil is needed during the scanning; the instantiation of this template specification may reference a laboratory equipment list that is dynamically updated to indicate, for each schedule time slot, whether a given ancillary equipment item is available, not-in-use (for example, because it is undergoing maintenance), or is assigned (and in the latter case to which imaging examination it is assigned). Similar assignment lists may be dynamically maintained for various types of staff members (where the categories for each time slot and for each staff member may be available, off-duty, or assigned to a particular imaging examination). These staff assignment lists may also tag staff members based on their respective qualifications, e.g. whether a staff member is qualified to administer a radiopharmaceutical to the patient, and these tags may be additionally used in assigning staff to specific imaging examination workflow phases. Similarly, a radiologist on-call list may be dynamically maintained and referenced to identify specific radiologist(s) available to review images if the template calls for radiologist review of the images prior to completion of the imaging examination. The various staff and radiologist lists may also include contact information (e.g. cellphone numbers, email addresses), so that alerts (e.g., via instant messaging, i.e. IM, and/or email) can be automatically sent to appropriate personnel prior to commencement of a next phase of an imaging examination.

In some embodiments, the patient-specific instantiation may also include referencing the patient's electronic medical record (EMR) or other patient database to identify patient-specific information used in instantiating the patient-specific workflow. For example, the EMR may be referenced to identify patient gender, weight, drug allergies, or the like, which may be relevant in sizing equipment to the patient, selecting from a set of suitable radiopharmaceuticals or contrast agents, or so forth.

During the examination, the system updates the visualization of the workflow based on available data. For example, the current time may be marked. If a procedure is delayed, then the visualization is updated to reflect this. In a manual embodiment, such an update may be done by a clerical worker using a GUI to adjust the times. On the other hand, if the system is receiving automatically generated data (for example, monitoring the hospital network for relevant Health Level 7 (HL7) messages (or messages conforming with another standard format used at the hospital), receiving and analyzing video from tracking cameras, radio frequency identification (RFID) tag readings used in a real-time locating system (RTLS) implemented at the hospital, status information received from the imaging device itself, and/or so forth) then this can be used to automatically update the visualization of the workflow.

In some embodiments, the system may further output alerts or reminders to medical personnel when it is detected (based on the automatically generated data) that a certain phase is reached (or will soon be reached). For example, if the system monitors the MRI scanner then it may thereby determine precisely when the imaging scan will be finished, and can issue an alert to patient transport personnel (e.g. via IM and/or email as previously mentioned) a few minutes ahead of the end of the scan. In a more advanced embodiment, the system correlates workflows of different patients, so that for example if a first patient will be delayed in entering the scan room then a second patient may be moved ahead of the first patient.

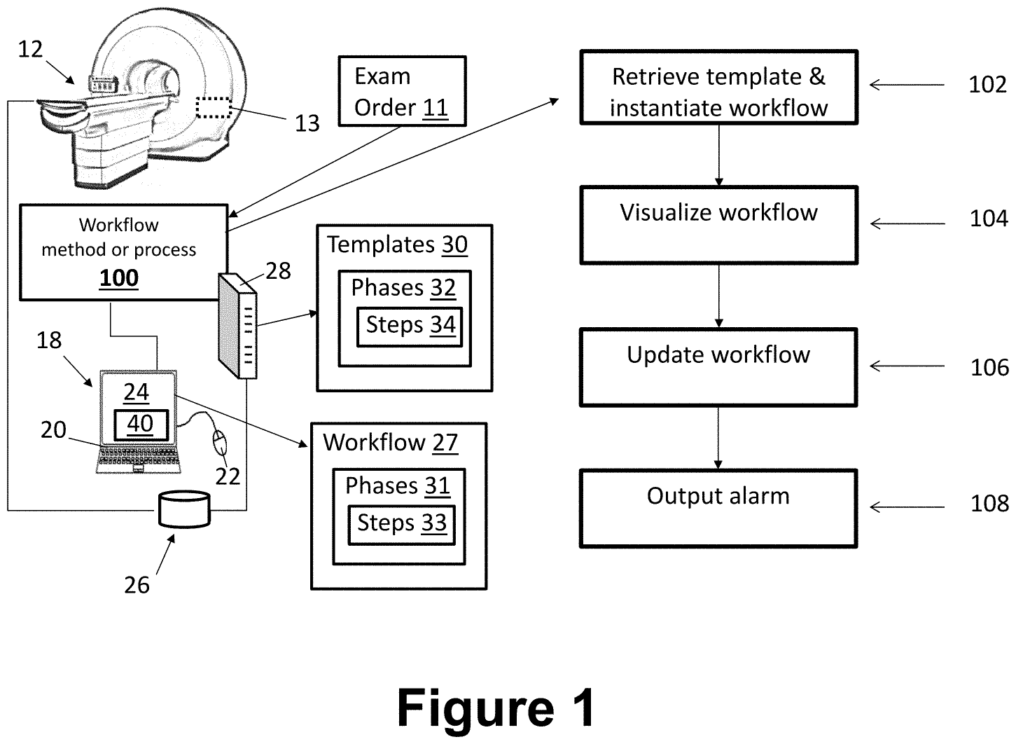

With reference to FIG. 1, an illustrative apparatus 10 for generating an imaging examination workflow is shown. The apparatus 10 receives an imaging examination order 11, and is used in conjunction with an image acquisition device (also referred to as an imaging device) 12. The image acquisition device 12 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 12 may also be a hybrid imaging device such as a PET/CT or SPECT/CT imaging system. While a single image acquisition device 12 is shown by way of illustration in FIG. 1, more typically a medical imaging laboratory will have multiple image acquisition devices, which may be of the same and/or different imaging modalities. For example, if a hospital does many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example.

FIG. 1 also shows an electronic processing device 18, such as a workstation computer, or more generally a computer. Alternatively, the electronic processing device 18 can be embodied as a server computer or a plurality of server computers, e.g. interconnected to form a server cluster, cloud computing resource, or so forth. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of an imaging examination workflow 27 for display on the display device 24.

The apparatus 10 also includes, or is otherwise in operable communication with, a database 28 storing a plurality of templates 30 for different types of imaging procedures or workflows 27. The database 28 can be any suitable database, including a Radiology Information System (RIS) database, a Picture Archiving and Communication System (PACS) database, an Electronic Medical Records (EMR) database, and so forth. Alternatively, the database 28 can be implemented in the non-transitory medium or media 26. The workstation 18 can be used to access the stored templates 30, along with relevant information of the current image exam in the database 28 (e.g., the scheduling information, the procedure description, the patient information, and so forth).

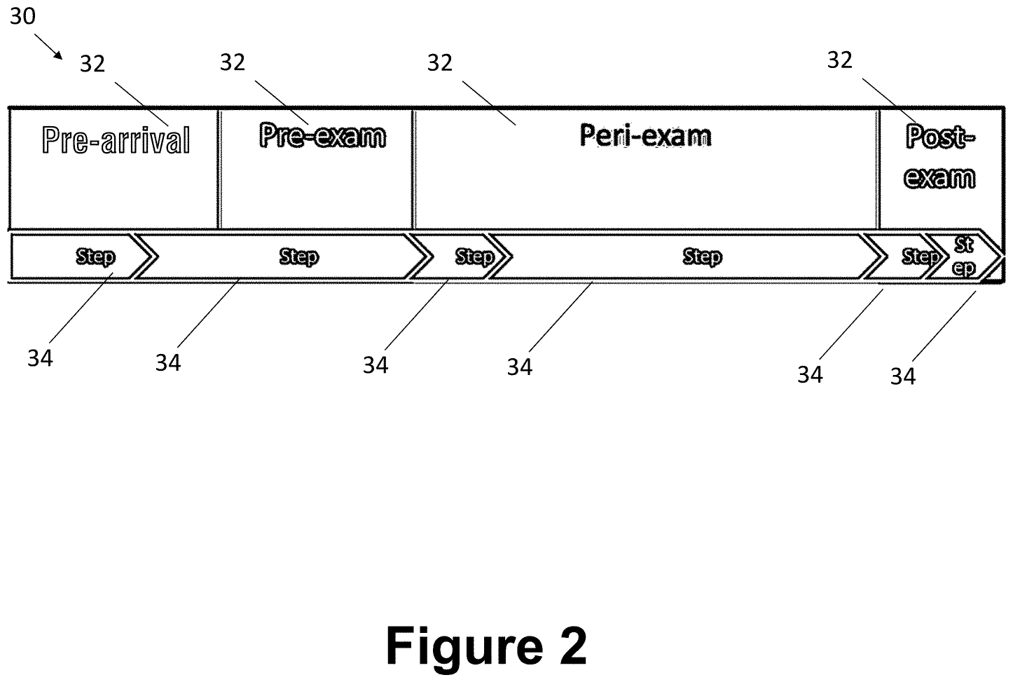

FIG. 2 shows an example of one of the templates 30. The templates 30 store a set of phases of the imaging procedure, personnel needed, equipment need, and so forth. Each template 30 is designed for a particular type of imaging examination. The type of imaging examination may be defined in terms of imaging modality, anatomy being imaged, contrast agent employed (if any), radiopharmaceutical employed (for emission imaging modalities), the imaging sequences to be performed, and/or so forth. The apparatus 10 retrieves the appropriate template for displaying a workflow for the examination order 11, based on the information contained in the examination order (such as the imaging modality, anatomy being imaged, or so forth), and optionally further based on information provided by an imaging technologist. As an example of the latter, upon receipt of the examination order 11 the apparatus 10 may identify one, two, or more templates that match the information contained in the examination order 11. If two or more matching templates are identified then the apparatus 10 may display a selection list of the matching templates, and a user then selects one of these matching templates using the selection list. The (automatically or semi-automatically) selected template is then instantiated to generate the workflow 27. The templates 30 include predicted time windows for the workflow 27. In the template, the time windows are typically given as time intervals relative to a reference time (e.g., time t=0 may be defined as the start of the first phase of the template. To instantiate the workflow 27 from the template, these time intervals are converted to actual times referenced to the scheduled start time for the workflow 27 in the examination schedule.

The templates 30 can be in any suitable format, including a BPMN model, a XML model, and so forth. The template 30 includes one or more phases 32. The phases 32 include time-duration events in the imaging workflow (of the type represented by the template 30), such as patient pre-arrival, room preparation, in-room events, post-imaging events, and so forth. Each phase 32 may include one or more steps 34. For example, a “patient pre-arrival” phase 32 can include steps 34 such as ensuring the patient has completed necessary pre-appointment criteria (e.g., fasting for a required amount of time, taking required medication before the appointment, and so forth). a “pre-exam” phase 32 can include steps 34 such as patient registration, patient/room preparation, wait times, and so forth. A “peri-exam” phase 32 can include in-room steps 34 such as positioning of the patient for imaging, performing the imaging procedure, and so forth. A “post-exam”phase 32 can include post-imaging steps 34 such as wait times, image processing, review of the images with the patient, and so forth. Each step 34 is associated with a duration, the resources and the events, and can contain a list of actions to be taken by actors in a context. These are merely non-limiting examples, and should not be construed as limiting. In addition, each template 30 can include similar phases 32 and/or steps 34.

Each workflow template 30 is defined by factors such as imaging modality (e.g., CT, MR, X-ray, mammography, US, and so forth); a body part or anatomy to be imaged (e.g., a neurological imaging, musculoskeletal imaging, chest imaging, abdomen imaging, and so forth); a reason for exam; a workflow criteria (a stat order, screening, diagnosis, biopsy, pre-operative, post-operative, follow-up, pre-discharge, and so forth); patient acuity (e.g., foreign body, allergy to contrast, allergy to sedation, claustrophobia, build, health literacy, and so forth), or so forth. In one example, the duration of the steps 34 can be configured in any suitable manner including static, configured based on the protocol, the exam information, or facility-specific prior performance data. In another example, the duration of the steps 34 can be dynamically adjusted, according to time of the day, the day of the week, the expertise and workload of the technologist on duty, and completion of prior events. For example, if a step 34 requires 90 minutes to complete, and there is only 60 minutes left in a workday, then the step can be rescheduled for the next day. In another example, if a technologist having a required amount of expertise is needed for a particular exam, and the technologist has no more availability in the day (or is not available until later in the day), then the workflow 27 can be updated to place this exam on the technologist's schedule for the next day (or later in the current day). In a further example, if a holiday is in the middle of a work week where the medical facility would be closed, the steps 34 (e.g., fasting procedures) can be updated for completion on the next workday. In addition, staff schedules can be updated similarly. Moreover, the workflow 27 can be updated to accommodate emergency procedures. For example, if there is a multi-car wreck, and the people from the accident are transferred to the medical facility, then the workflow 27 can be updated to move or otherwise accommodate equipment and personnel to treat the patients.

Instantiating the template for the imaging examination order 11 can trigger an initiation of the phases 32 and/or steps 34 as appropriate for the particular examination order 11. For example, if the examination order 11 calls for additional imaging not included in the template 30, then a (sub)template for performing that additional imaging may be inserted into the image acquisition phase of the template. Before and during the performance of the imaging examination, the workflow 27 is displayed on the display device 24, including displaying the various phases and steps. The display is preferably a plot of the workflow phases as a function of time (i.e., the abscissa is time). The display of the workflow 27 (and possibly the workflow 27 itself) is updated in real-time in accordance with the progress of the examination. For example, a marker may be displayed which indicates the current point in the workflow, e.g. marking when the registration step 34 is complete, when the patient is in the waiting room, and so forth.

The apparatus 10 is configured as described above to perform an imaging examination workflow visualization method or process 100. The non-transitory storage medium 26 stores instructions which are readable and executable by the at least one electronic processor 20 to perform disclosed operations including performing the imaging examination workflow visualization method or process 100. In some examples, the method 100 may be performed at least in part by cloud processing.

Referring back to FIG. 1, an illustrative embodiment of imaging examination workflow visualization method 100 is diagrammatically shown as a flowchart. At an operation 102, the template for the examination order 11 is retrieved. As previously mentioned, if two or more different templates 27 match the exam order 11, then a user dialog such as a selection list is displayed which lists the matching templates and allows the user to select the template to be retrieved (e.g., using the at least one user input device 22, such as a mouse, trackball, touch-sensitive display, or other pointing device).

The operation 102 further entails instantiating at least one workflow 27 from the retrieved template 30. This includes mapping start and end times of the phases to the schedule (e.g., by mapping the default “zero” time of the template to an actual schedule time), filling in information for the phases using dynamically updated imaging scanners, laboratory equipment and/or personnel lists, or so forth. The instantiation of the template 30 to form the workflow 27 may further include updating the retrieved template based on information contained in the examination order 11 and/or retrieved from the patient's EMR record. For example, if information retrieved from a particular patient's EMR record indicates that the patient has a medical device implant, then one or more steps 34 can be added to the workflow 27 for that patient. In another example, the workflow 27 can be updated for a patient requiring an intravenous (IV) device to allow time to attach the IV device to the patient. In a further example, patients having conditions or an age that results in diminished movement, the workflow 27 can be updated to include extra time for transporting the patient.

In one example, the at least one electronic processor 20 generates the at least one workflow 27 by modifying the template 30 based on a user input provided by a user via the at least one user input device 22 of the workstation 18. The modifications to the template 30 can include one or more of: filling in scheduled time for completion of one or more of the steps 34 and phases 32 of the template (this allows for manual adjustment of time intervals); adding or removing phases from the template (for example, if the examination order 11 specifically indicates a contrast agent be used which is not included in the template, then a contrast agent administration phase may be added); adding one or more sub-templates to the at least one workflow (for example, if the patient is wheelchair-bound then a sub-template for wheelchair transport and transfer of the patient from the wheelchair to the table and vice versa may be added); and adding patient-specific information to the phases. In another example, if a patient has not completed necessary pre-appointment criteria (e.g., fasting, medication, etc.), then the template 30 can be modified to update the workflow 27 to re-schedule the patient. In some embodiments, the workflow 27 can be generated by modifying the template 30 to recapture time lost during the day. For example, a schedule of one or more staff members can be adjusted so that the staff members can help with a log jam of exams. In addition, allocation of devices can be changed to help with such a log jam. In another example, the schedule of staff members can be adjusted so that more experienced staff members can assist with longer and/or more complicated exams, while the schedules of the less-experienced staff members can be modified to assign them to simpler, less time-consuming procedures.

The at least one workflow 27 corresponding to the imaging examination work order 11 for a patient is displayed on the display device 24 in an operation 104. The at least one workflow 27 includes a set of phases 31 over predicted time windows for the workflow, and each phase in the set of phases may include one or more steps 33 to be performed in the workflow.

The visualization operation 104 is also updated in real time as the imaging examination progresses. For example, completed phases 32, the current phase, and not-yet-reached phases may be depicted in different ways (e.g., completed phases may be greyed out, the current phase may be shown in boldface, and not-yet-reached phases may be shown in regular font; but this is merely an example). Optionally, the visualization of the current phase 32 may include textual and/or graphical annotations indicating relevant information such as any ancillary equipment that is in use, whether imaging data acquisition is presently occurring (for example, the apparatus 10 may monitor the imaging device 12 to determine this information in real time, and the current phase 32 of the workflow 27 may be highlighted in red or otherwise annotated to indicate ongoing imaging data acquisition), and/or so forth.

At an optional operation 106, the workflow 27 is updated based on ongoing events during the imaging examination. For example, if there are unexpected delays, then the time intervals for the phases 32 may be adjusted. If the patient is expected to be wheelchair-bound but arrives otherwise, then phases 32 related to wheelchair transport may be removed. Similarly, the imaging technician may add or remove imaging sequence phases 32 based on what is observed in the initial imaging sequences. In addition, the workflow 27 can be updated if a time slot opens (e.g., a patient is rescheduled for not fasting as required before an examination).

In the operation 106, the at least one electronic processor 20 is programmed to dynamically update the at least one workflow 27 on the display device 24 with additional data related to one or more steps 33 of the phases 31 of the at least one workflow. To do so, the at least one electronic processor 20 is programmed to modify the template 30 based on currently available information for the imaging examination order 11. The modifications to the template 30 can include one or more of: updating a scheduled time for completion of one or more of the steps 36 and phases 34 of the template; adding or removing phases from the template; adding one or more sub-templates to the at least one workflow; adding patient-specific information to the phases; and adding one or more of historical patient data, present workload, and staff levels to adjusting the predicted time intervals for one or more of the phases 31.

In a further example of the operation 106, the currently-available information includes a current time, and the at least one electronic processor 20 is programmed to dynamically update the at least one workflow 27 by automatically marking the current time in the workflow (e.g., via the at least one user input device 22), and updating the workflow on the display device 24 based on the marking of the current time.

In yet another example of the operation 106, the at least one electronic processor 20 is programmed to monitor information from one or more of: a hospital network for relevant messages, analyzing video from one or more tracking cameras, RFID tag readings, status information received from a medical device, and so forth. From this monitored information, the at least one electronic processor 20 is programmed to generate the currently available information to update the at least one workflow 27.

In another example of the operation 106, the at least one electronic processor 20 is programmed to monitor a controller 13 of the imaging device 12 used in performing the imaging examination order 11 for the patient. The additional data used in the dynamic updating of the at least one workflow 27 includes additional data generated by the monitoring of the controller 13 of the imaging device 12.

It is noted that the dynamic updating operation 106 is optional. In some contemplated embodiments, the dynamic updating operation 106 is not provided. In these embodiments, the visualization is still improved over existing workflow displays in that the workflow visualization is provided with a high degree of granularity, with that high granularity information specifically tailored to the specific imaging examination order 11 and to the specific patient.

At an optional operation 108, the at least one electronic processor 20 is programmed to generate an alert or reminder 40 when one or more of the steps 33 of the phases 31 of the at least one workflow 27 is completed or will soon be completed. In one example, the at least one electronic processor 20 is programmed to correlate a plurality of workflows 27 corresponding to multiple patients using the predicted time windows of corresponding phases 31 of each workflow, and output one or more alerts or reminders 40 when the predicted time windows of the multiple workflows are in conflict. The alerts or reminders 40 can be output visually (e.g., on the display device 24) or audibly (e.g., via a loudspeaker which is not shown). Suitable alerts 40 can also be issued a suitable time before a phase ends. For example, at, e.g., 3 minutes before the scheduled end of the imaging data acquisition phase IM and/or email alerts may be sent to the on-call transport staff informing them that the patient will be ready for transport in 3 minutes. In some examples, an alert or reminder 40 can be generated when an actual duration of the phases 31 and/or steps 31 exceed an expected duration as defined in one of templates 30.

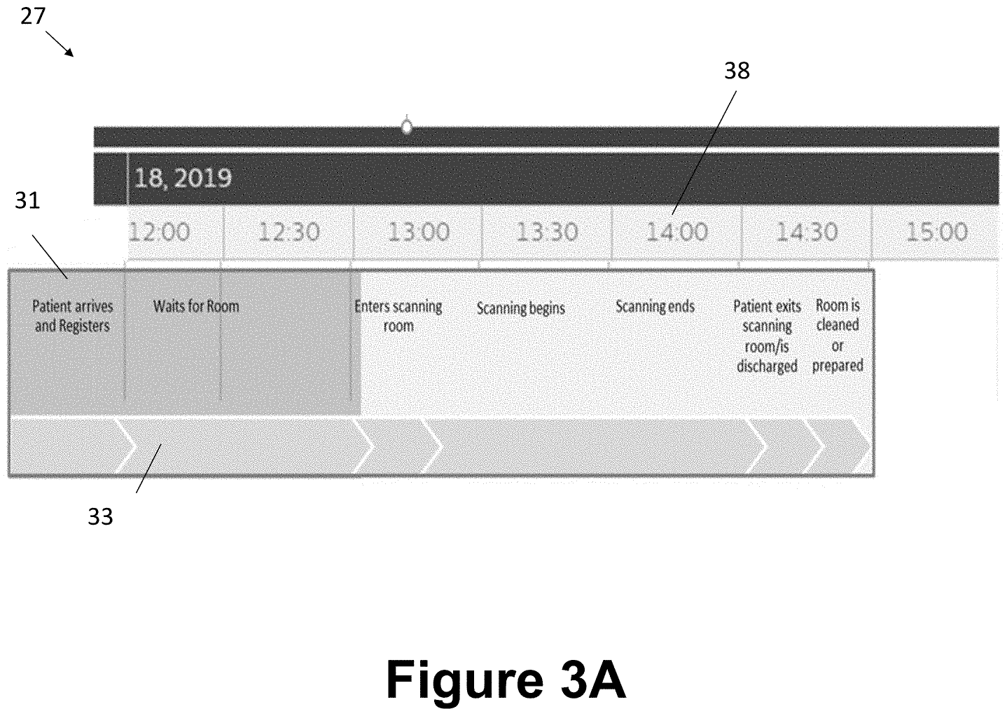

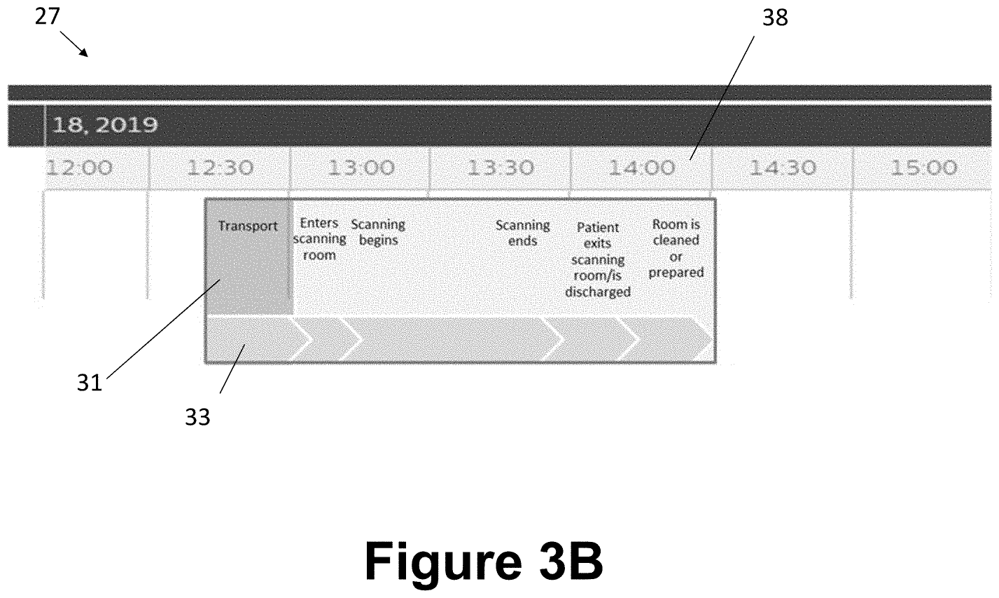

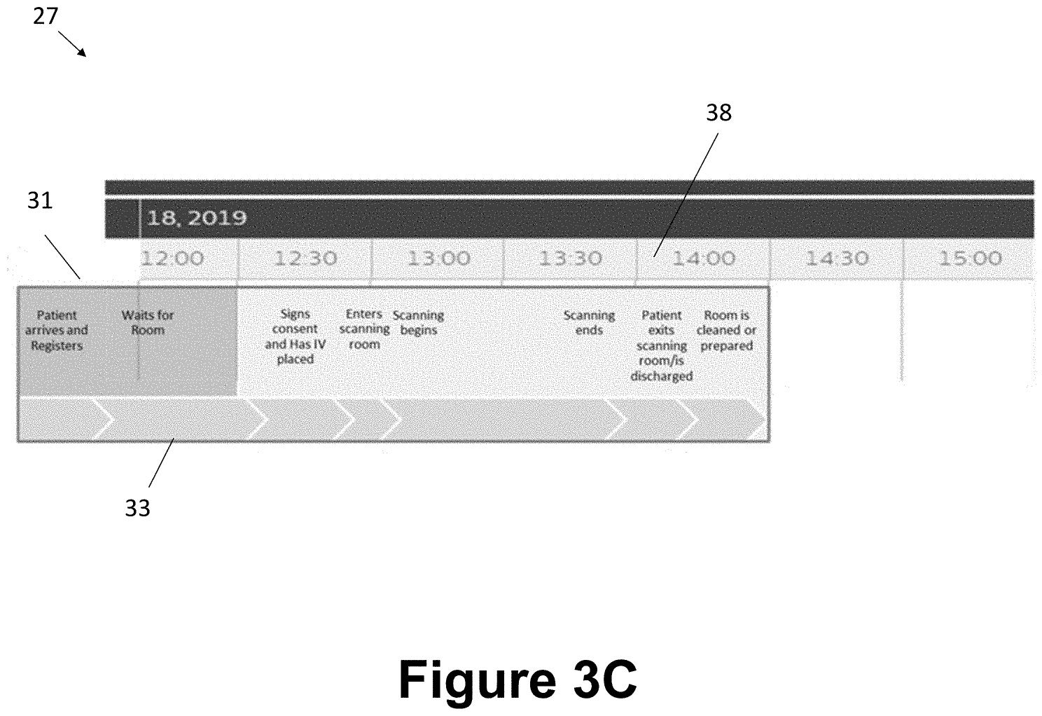

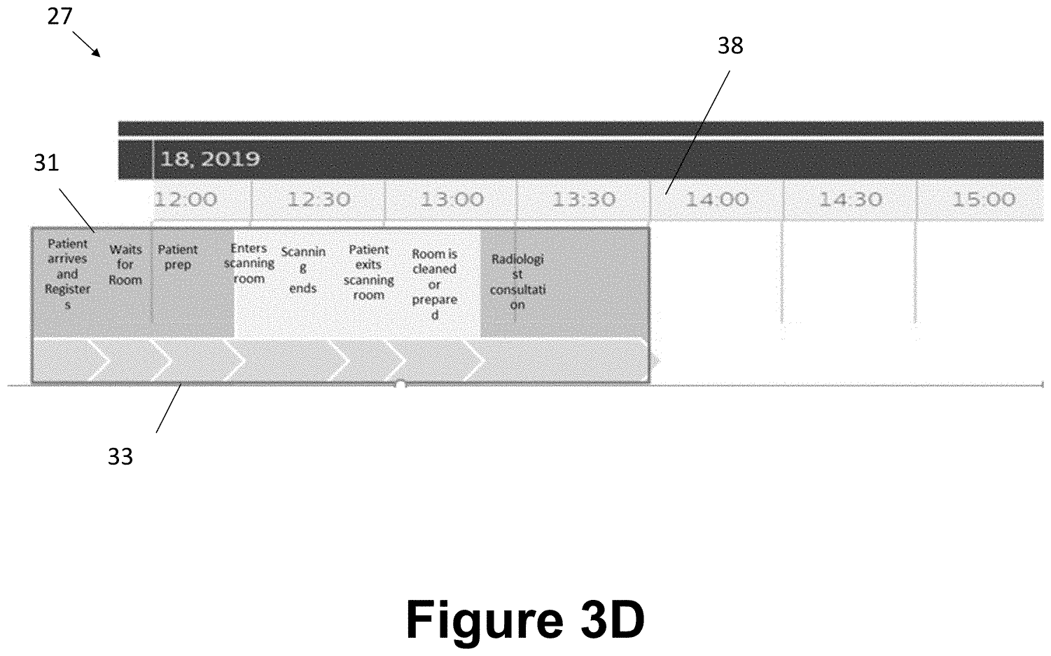

FIGS. 3A-3D show examples of workflows 27 as displayed on the display device 24. It will be appreciated that the workflows 27 shown in FIGS. 3A-3D can also be similarly shown as the templates 30. As shown in FIGS. 3A-3D, each workflow 27 includes respective phases 31 and steps 33 (or, similarly, phases 32 and steps 34 of a template 30), and also shows time windows 38. FIG. 3A shows a workflow 27 for a MR without contrast outpatient workflow. FIG. 3B shows a workflow 27 for a CT without contrast inpatient workflow. FIG. 3C shows a workflow 27 for a CT without contrast outpatient workflow. FIG. 3D shows a workflow 27 for a mammography screening.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An apparatus, comprising:
at least one electronic processor programmed to:
display, on a display device, at least one workflow corresponding to an imaging examination order for a patient, the workflow including a set of phases over predicted time windows for the workflow, each phase in the set of phases including one or more steps to be performed in the workflow; and
dynamically update the at least one workflow on the display device with additional data related to one or more steps of the phases of the at least one workflow.

2. The apparatus of claim 1, wherein the at least one electronic processor is further programmed to retrieve a template for a type of imaging examination to which the imaging examination order corresponds, the at least one workflow comprising or being based on the template.

3. The apparatus of claim 2, further comprising:
at least one user input device,
wherein the at least one electronic processor is programmed to generate the at least one workflow by modifying the template based on user input received via the at least one user input device.

4. The apparatus of claim 3, wherein the modifying to generate the at least one workflow includes one or more of:
filling in scheduled time for completion of one or more of the steps and phases;
adding or removing phases from the template;
adding one or more sub-templates to the at least one workflow; and
adding patient-specific information to the phases.

5. The apparatus of claim 1, wherein the at least one electronic processor is programmed to dynamically update the at least one workflow on the display device with additional data related to one or more steps of the phases of the at least one workflow by:
modifying the template based on currently available information for the imaging examination order.

6. The apparatus of claim 5, wherein the modifying includes one or more of:
filling in updated scheduled time for completion of one or more of the steps and phases;
adding or removing phases from the at least one workflow;
adding one or more sub-templates to the at least one workflow; and
adding patient-specific information to the phases.

7. The apparatus of claim 5, wherein the modifying includes:
adding one or more of historical patient data, present workload, and staff levels to adjusting the predicted time intervals for one or more of the phases.

8. The apparatus of claim 2, further including:
a database storing a plurality of templates for different types of imaging procedures, the templates storing a set of phases of the imaging procedure, personnel needed, and equipment need.

9. The apparatus of claim 8, wherein the templates are a BPMN model or a XML model.

10. The apparatus of claim 5, wherein the currently available information includes a current time and the at least one electronic processor is programmed to dynamically update the at least one workflow on the display device with additional data related to one or more steps of the phases of the at least one workflow by:
automatically marking the current time in the at least one workflow; and
updating the at least one workflow on the display device based on the marking of the current time.

11. The apparatus of claim 5, wherein the electronic processor is further programmed to:
monitor information from one or more of a hospital network for relevant messages, analyzing video from one or more tracking cameras, RFID tag readings, status information received from a medical device; and
generate the currently available information from the monitored information.

12. The apparatus of claim 1, wherein the at least one electronic processor is programmed to dynamically update the at least one workflow on the display device with additional data related to one or more steps of the phases of the at least one workflow by:
receiving, via at least one user input device, a marking of a current time in the at least one workflow; and
updating the at least one workflow on the display device based on the marking of the current time.

13. The apparatus of claim 1, wherein the electronic processor is further programmed to:
monitor a controller of an imaging device used in performing the imaging examination order for the patient;
wherein the additional data used in the dynamic updating includes additional data generated by the monitoring of the controller of the imaging device.

14. The apparatus of claim 1, wherein the electronic processor is further programmed to:
output one or more alerts or reminders when one or more of the steps of phases of the at least one workflow is completed or will soon be completed.

15. The apparatus of claim 14, wherein:
correlate a plurality of workflows corresponding to multiple patients using the predicted time windows of corresponding phases of each workflow;
output the one or more alerts or reminders when the predicted time windows of the multiple workflows are in conflict.

16. An apparatus, comprising:
at least one electronic processor programmed to:
display, on a display device, at least one workflow corresponding to an imaging examination order for a patient, the workflow including a set of phases over predicted time windows for the workflow, each phase in the set of phases including one or more steps to be performed in the workflow; and
retrieve a template for a type of imaging examination to which the imaging examination order corresponds, the at least one workflow comprising or being based on the template.

17. The apparatus of claim 16, further comprising:
at least one user input device,
wherein the at least one electronic processor is programmed to generate the at least one workflow by modifying the template based on user input received via the at least one user input device.

18. The apparatus of claim 17, wherein the modifying to generate the at least one workflow includes one or more of:
filling in scheduled time for completion of one or more of the steps and phases;
adding or removing phases from the template;
adding one or more sub-templates to the at least one workflow; and
adding patient-specific information to the phases.

19. The apparatus of claim 17, further including:
a database storing a plurality of templates for different types of imaging procedures, the templates storing a set of phases of the imaging procedure, personnel needed, and equipment need.

20. A method, comprising:
displaying, on a display device, at least one workflow corresponding to an imaging examination order for a patient, the workflow including a set of phases over predicted time windows for the workflow, each phase in the set of phases including one or more steps to be performed in the workflow;
retrieving a template for a type of imaging examination to which the imaging examination order corresponds, the at least one workflow comprising or being based on the template;
dynamically updating the at least one workflow on the display device with additional data from the retrieved template; and
outputting one or more alerts or reminders when one or more of the steps of phases of the at least one workflow is completed or will soon be completed based on the dynamically updated at least one workflow.
